(19) United States
(12) Patent Application Publication
(10) Pub. No.: US 2010/0112611 A1
(43) Pub. Date: May 6, 2010

(54) PROCEDURE FOR DETECTING MICROBIAL CONTAMINATION BY BIOLUMINESCENCE IN ASSOCIATIVE ACRYLIC THICKENERS AND PRODUCTS CONTAINING THEM

(75) Inventors: Henri GRONDIN, Sathonay Village (FR); Sylvie Leveque, Neuville Sur Saone (FR); Mehdi Bouzid, Rock Hill, SC (US)

Correspondence Address: OBLON, SPIVAK, MCCLELLAND MAIER & NEUSTADT, L.L.P., 1940 DUKE STREET, ALEXANDRIA, VA, 22314, US

(73) Assignee: COATEX S.A.S., Genay (FR)

(21) Appl. No.: 12/611,582

(22) Filed: Nov. 3, 2009

Related U.S. Application Data
(60) Provisional application No. 61/112,914, filed on Nov. 10, 2008.

(30) Foreign Application Priority Data
Nov. 3, 2008 (FR) 08 06088

(51) Int. Cl. C12Q 1/66; C12Q 1/02

(52) U.S. Cl. 435/8; 435/29

(57) ABSTRACT

A procedure for detecting microorganisms by bioluminescence in an aqueous formulation containing an ASE or HASE-type polymer, which implements at least one step of dilution of the aqueous formulation. This dilution step, and notably the regulation of the dilution factor, allows the bioluminescence technique, up to now ineffective on this type of products, to be implemented. It can henceforth be used on these ASE or HASE-type polymers, but also on products containing them, such as a paper coating, paint, lacquer, varnish or stain.

### CROSS-REFERENCE TO RELATED APPLICATIONS

The present application claims priority to U.S. 61/112,914, filed on Nov. 10, 2008, and French Application No. 08 06088, filed on Nov. 3, 2008. The entire contents of these applications is incorporated herein by reference.

### FIELD OF THE INVENTION

The present invention concerns a procedure for detecting microbial contamination by ATP-metry, especially intended for associative acrylic polymers of ASE and HASE type and various products in which the said polymers can be found. This procedure makes it now possible to use ATP-metry, which relies on an optimal enzymatic bioluminescence reaction at a near-neutral pH, to detect contamination in these ASE and HASE-type polymers.

### BACKGROUND OF THE INVENTION

While in the past, standard tests of incubation in Petri dishes have been performed, with a duration sometimes greater than one week, the Applicant has defined strict conditions under which microbial contamination can be detected in an almost immediate and optimal manner for these special polymers, both as is and in the various formulations that may contain them such as a paint, using the invention.

In the context of industrial activities such as the manufacture of chemical products, inspection for microbial contamination is currently subject to strict requirements. Having available high-performance tools for detection of any microbial contamination in an optimal and rapid manner is a priority: under these terms, those in the chemical industry must now guarantee rigorous monitoring of the quality of the products manufactured, internally and to their customers.

Conventionally, microbiological follow-up has been performed after collection of a sample before the product is delivered to the customer. The said sample was cultured on a specific agar, in a Petri dish and under standardised temperature conditions. The test was positive when colonies of microorganisms visible to the naked eye appeared: this took at least 24 hours, and for the ASE or HASE-type products concerned, from 3 to 7 days.

For faster detection, there is an alternative approach based on the metabolic activity of the microorganisms: this method allows the minimum duration necessary for their culture in agar to be avoided. This technique is known as ATP-metry and relies on the phenomenon of bioluminescence. Its principle rests on the determination of the level of adenosine triphosphate (ATP), a universal metabolic marker and thus present in all living cells. As an example, it is known (see the document http://bioluminesecence.free.fr/dosatp.htm) that a bacterium of the Proteus Vulgaris type has an average ATP level on the order of 0.18 femtogramme (fg) per cell.

ATP-metry is widely used today to rapidly reveal the presence of microorganisms in numerous fields of activity, as in industrial process water effluents (U.S. Pat. No. 4,385,113), in pharmacy, cosmetics, and electronics (U.S. Pat. No. 5,366,877, U.S. Pat. No. 5,766,868), in blood and urine (U.S. Pat. No. 3,971,703), and even very generally in paints (WO 04 078 997).

As described in the document “European Pharmacopoeia 6.0” (section 5.1.6 pp. 571-572), this method consists of stimulating the release of ATP by microorganisms using a suitable extraction agent, then performing a quantitative determination of this by means of the luciferin-luciferase enzymatic system: a quantity of light, measured by a luminometer and expressed in relative light units (RLU), is then emitted, proportional to the quantity of ATP present. In this way the presence of microorganisms in the sample analysed can be detected extremely rapidly.

High-performance portable systems, in the form of pens containing a removable probe or swab for sample collection, are now manufactured. The probe is put back into place in the pen. It is then plunged into an extracting and buffering medium allowing the ATP present in the cells to be released and the medium to be simultaneously kept at a near-neutral pH. This mixture then comes into contact with a tablet containing the luciferin/luciferase system: this is when the bioluminescence reaction takes place.

After a few moments, the pen is inserted directly into the luminometer, which in a few seconds indicates the quantity of light emitted: any presence of microorganisms is thus detected. This type of device is notably described in the document U.S. Pat. No. 5,917,592. It is also illustrated in the document U.S. Pat. No. 5,965,453: the presence of cavity 32, in FIG. 2, which serves to accommodate the buffer system, can be noted.

As is well known by professionals in the field, the presence of a buffer system is an essential element in measurement by ATP-metry. In fact, a near-neutral pH corresponds to the optimal range for the bioluminescence reaction which makes use of the luciferin/luciferase pair. In this regard, one can refer to the document WO 94/11528, which shows that the optimal pH for the bioluminescence reaction lies between 7.7 and 7.8 (page 1, lines 18-20), and the document WO 95/04276, which proposes the use of a buffer to set the pH at a value lying between 7 and 8 (page 3, lines 22-28). The document U.S. Pat. No. 7,132,249, which proposes working in a pH range between 6.4 and 7.2 (claim 17), can also be cited.

However, in the area of chemistry there exist products that are a priori incompatible with the use of bioluminescence: these products actually gel in the aqueous phase when they are placed under near-neutral pH conditions. This is all the more unfortunate as such products are widely exposed to a risk of microbial contamination, insofar as they are formulated in the presence of a large quantity of water. Moreover, they are used in compositions which are themselves aqueous formulations susceptible to contamination, such as paints, coating mixtures, and cosmetic or detergent formulations. Finally, these products are very advanced technical solutions, and it is regrettable that they cannot benefit from the progress in detection of microbial contamination by bioluminescence.

These products are thickeners of the ASE and HASE type. The term thickener designates a category of compounds which, when introduced into an aqueous medium, are capable depending on certain conditions of increasing the viscosity: they thicken the medium by gelling the aqueous phase. Thickeners of ASE (Alkali Swellable Emulsion) type designate emulsion thickeners that are copolymers of (meth)acrylic acid with an ester of these acids, while thickeners of HASE (Hydrophobically modified Alkali Swellable Emulsion) type designate emulsion thickeners that are copolymers based on (meth)acrylic acid, an ester of these acids and a monomer with a hydrophobic group.

The mechanisms of action of these products differ. The polymers of ASE type, in the form of dispersions in the acidic state, only become soluble in the neutral state. When the medium is neutralised, an ionic repulsion mechanism is induced between the various carboxylate groups on the polymeric chain. These ionised groups polarise a large number of water molecules, leading to the increase in viscosity of the medium. In addition to the aforementioned ionic polarising phenomenon, the HASE-type polymers involve interactions between their hydrophobic groups, which also contribute to thickening the medium.

These mechanisms, and especially the ability to thicken an aqueous medium at near-neutral pH, have been described in the documents WO 2007/144721 and “Practical guide to associative thickeners” (Proceedings of the Annual Meeting Technical Program of the FSCT (2000), 78th, 644-702). Numerous applications for these thickeners are found in paints, coating mixtures, or cosmetics (FR 2 693 203, FR 2 872 815, FR 2 633 930, FR 2 872 815).

In practical terms, it is impossible to use the aforementioned detection systems based on pens in the case of ASE or HASE-type polymers or in a formulation containing them; as soon as these polymers come into contact with the buffer system, gelling of the medium occurs. The system is then congealed and can no longer come into contact with the luciferin/luciferase complex: the bioluminescence reaction cannot occur.

Therefore, there is a very strong prejudice for the professional specialising in the ASE and HASE-type polymers against using bioluminescence for detecting microbial contamination in this type of products: he considers it a fundamental principle that this type of thickener cannot be formulated and transported unless it is kept in an acidic medium, without which it thickens by gelling the aqueous phase.

### DESCRIPTION OF THE INVENTION

However, in seeking to develop an extremely rapid system for detection of microorganisms in an ASE or HASE-type thickener or in a product containing them, the Applicant has overcome the prejudice against the bioluminescence method. He has managed to develop strict rules which can be applied to detect the presence of microorganisms in an ASE or HASE-type polymer, or in a product containing them, by ATP-metry. These rules are based in particular on a very precise principle of dilution.

In this regard, an initial objective of the invention consists of a procedure for detection of microorganisms by bioluminescence in an aqueous formulation containing an ASE or HASE-type polymer, characterised by the fact that it makes use of at least one step of dilution of the said aqueous formulation.

This procedure for detection of microorganisms by bioluminescence in an aqueous formulation containing an ASE or HASE-type polymer is also characterised by the fact that it includes:

a) at least one step of dilution of the said aqueous formulation containing an ASE or HASE-type polymer,

b) a step bringing the formulation resulting from step a) into contact with at least one ATP-extracting compound and at least one pH-regulating compound,

c) a step bringing the medium resulting from step b) into contact with a luciferin/luciferase system,

d) a step measuring the quantity of light emitted by bioluminescence by the medium resulting from step c).

This detection procedure is also characterised by the fact that the dilution factor of the said aqueous formulation in step a) lies between 5 and 300, preferably between 5 and 100, and most preferably between 5 and 20.

This procedure is also characterised by the fact that the extracting agent in step b) is chosen from among the organic solvents or surfactants such as trichloroacetic acid (TCA) or dimethyl sulfoxide (DMSO).

This procedure is also characterised by the fact that the pH-regulating agent in step b) is a buffer chosen from among the organic acids and bases, and notably the phosphate buffers such as potassium or sodium dibasic phosphate.

This procedure is also characterised by the fact that dilution step a) is carried out by mixture with a sterile aqueous solution, preferably a sterile isotonic aqueous solution, and most preferably a sterile isotonic aqueous peptone solution.

By isotonic, the Applicant means a salt concentration in solution on the order of 0.9 g/L.

Steps b), c) and d) are carried out according to the well-known methods of ATP-metry, a description of which can be found in the documents on the state of the technique already cited: U.S. Pat. No. 4,385,113, in U.S. Pat. No. 5,366,877, U.S. Pat. No. 5,766,868, U.S. Pat. No. 3,971,703, WO 04 078 997, WO 94/11528, U.S. Pat. No. 7,132,249.

Advantageously, this procedure is also characterised by the fact that it uses a luminometer having a probe containing:

1) an tip for collection of a sample of the formulation to be analysed,

2) an ATP-extracting compound,

3) a pH-regulating compound,

4) a luciferin/luciferase system.

This procedure is also characterised by the fact that the ASE-type polymer is a copolymer of (meth)acrylic acid with an ester of (meth)acrylic acid, and by the fact that the HASE-type polymer is a copolymer of (meth)acrylic acid, an ester of (meth)acrylic acid and a monomer having a hydrophobic group.

This procedure is also characterised by the fact that the said aqueous formulation is an emulsion, a dispersion or an aqueous composition containing a binder, this composition being chosen from among a paper coating, a paint, a varnish, an ink, a cosmetic or detergent composition, and in general any aqueous formulation containing an ASE or HASE-type thickener, notably those likely to be stored several days.

The following examples will allow the invention to be better understood, without however limiting its scope.

### EXAMPLES

In order to reproduce the present invention, the professional in the field can carry out tests either on already-contaminated or polluted products based on ASE or HASE-type polymers, or by artificially contaminating such products.

Reproduction of the invention does not consist of identical reproduction of the level of sample contamination as presented below. It consists of, for a given contamination, carrying out in particular the fundamental step of dilution of the product, which allows use of the bioluminescence technique; this dilution should preferably be performed within the limits set by the present invention.

### Example 1

This example aims to illustrate the implementation of the bioluminescence technique in accordance with the procedure of the present invention to reveal the presence of microorganisms in a HASE-type thickener deliberately contaminated for the purposes of the demonstration. This example demonstrates notably the effect of the dilution factor on the quality of the results, and illustrates the measurement speed compared to the standard test performed in a Petri dish.

A HASE-type associative thickener, as described in the document FR 2 693 203, is used.

This is a partially or totally water-soluble copolymer, made up of at least one ethylenically unsaturated monomer with a carboxylic function, and at least one ethylenically unsaturated oxyalkylated monomer and terminated by a hydrophobic fatty chain with at least 26 carbon atoms and possibly at least one monomer, at least doubly ethylenically unsaturated.

This type of product leads to a relatively strong thickener; in water at 6 g/L, a Brookfield™ viscosity lying between 1000 and 2000 mPa.s when measured at 25° C. and 100 rev/minute is obtained.

This thickener, in the form of an aqueous emulsion, is artificially contaminated (such contamination methods are notably described in the aforementioned document “European Pharmacopoeia 6.0”).

The sample is then diluted by a factor 0 (prior art), and by other factors (according to the invention), by mixture of the product to be tested with an aqueous isotonic sterile peptone solution.

The sample, more or less diluted, is then analysed by bioluminescence using a NovaLum II device commercialised by the CHARM SCIENCES INC. company.

This device consists in fact of a pen with a detachable probe, the end of which is used to collect a part of the sample to be analysed by immersion in this sample.

The probe is put back into place in the pen. It is then plunged into an extracting and buffering medium which simultaneously releases the ATP present in the cells and keeps the medium at a near-neutral pH.

This mixture, under gravity alone, then comes into contact with a tablet containing the luciferin/luciferase system.

The device also contains a mechanism capable of measuring the quantity of light emitted if the bioluminescence reaction has occurred: the pen is inserted into this device and a measurement in RLU is read.

Table 1 indicates the value in RLU of the quantity of light emitted at different levels of dilution. Several tests have been carried out at the same levels, so as to evaluate the reproducibility of the measurement: this is determined by means of the relative standard deviation, or the ratio between the standard deviation and the mean.

At zero dilution, measurement is impossible as, when the formulation comes into contact with the buffer, there is gelling of the medium. Consequently, the said medium can no longer flow and come into contact with the luciferin/luciferase system: the bioluminescence reaction cannot take place.

On the other hand, it is noted that higher dilution levels allow the bioluminescence reaction to proceed. The optimal range for detection, corresponding to the best measurement reproducibility, corresponds to a relative standard deviation equal to 0.18, obtained for a dilution factor between 5.5 and 10.

TABLE 1

| | Dilution | | | | | | | | |
|---|---|---|---|---|---|---|---|---|---|
| | 960 | 480 | 240 | 90 | 10 | 5.5 | 2.8 | 2.0 | 0 |
| Relative | 212,502 | 953,916 | 1,136,199 | 2,224,048 | 19,330,274 | 25,184,720 | 25,241,952 | 622,618 | Measure- |
| quantity | | | | | | | | | ment |
| of light | | | | | | | | | impossible |
| (RLU) | 177,259 | 352,423 | 1,019,314 | 1,350,359 | 15,690,027 | 18,002,348 | 16,646,289 | 123,470 | — |
| | 102,056 | 571,768 | 529,215 | 2,175,968 | 1254 909 | 19,640,556 | 3410 968 | 9,092,725 | — |
| | 277,663 | 571,240 | 756,770 | 1,757,399 | 12,608,826 | 16,938,826 | 8,756,299 | 895,791 | — |
| | 288,926 | 421,283 | 821,711 | 1,249,332 | 11,599,356 | 18,345,456 | 9,789,365 | 36,542,904 | — |
| | 272,265 | 432,861 | 734,393 | 1,201,929 | 13,747,784 | 23,456,756 | 12,832,546 | 36,853,088 | — |
| | 529,087 | 651,145 | 1,214,882 | 1,435,892 | 13,687,317 | 26,345,582 | 17,342,789 | 38,040,808 | — |
| Mean | 265,679 | 564,948 | 887,497 | 1,627,846 | 14,173,356 | 21,130,606 | 17,815,744 | 17,453,057 | — |
| Standard | 133,871 | 200,858 | 244,824 | 430,349 | 2,614,709 | 3,794,230 | 9,067,128 | 18,673,757 | — |
| deviation | | | | | | | | | |
| Relative | 0.5 | 0.36 | 0.28 | 0.26 | 0.18 | 0.18 | 0.51 | 1.07 | — |
| standard | | | | | | | | | |
| deviation | | | | | | | | | |

With a test carried out on the same contaminated sample using a Petri dish put into an incubator at 30° C., according to the method well known to professionals in the field, formation of colonies of microorganisms begins to be visible to the naked eye at the end of 3 days.

The advantage of implementing the method according to the invention, allowing sample contamination to be revealed almost immediately, is thus demonstrated.

### Example 2

This example aims to illustrate the use of the bioluminescence technique in accordance with the procedure of the present invention to reveal the presence of microorganisms in a HASE-type thickener deliberately contaminated for the purposes of the demonstration. This example demonstrates notably the effect of the dilution factor on the quality of the results.

A HASE-type associative thickener, as described in the document FR 2 872 815, is used.

This consists of a water-soluble acrylic copolymer made up of at least one ethylenically unsaturated monomer with a carboxylic function, at least one non-ionic ethylenically unsaturated monomer, and at least one ethylenically unsaturated oxyalkylated monomer terminated by a hydrophobic branched non-aromatic chain including from 10 to 24 carbon atoms.

This type of product leads to a more moderate thickener than the preceding one; put in water at 20 g/L, a Brookfield™ viscosity lying between 80 and 160 mPa.s is obtained measured at 25° C. and 100 rev/minute. It also gives an excellent resistance to salts in the case of a paint.

The procedure is identical to that described in test 1; for different dilution levels, the relative quantity of light emitted when the bioluminescence reaction occurs is determined. The results appear in Table 2.

Once again, the bioluminescence reaction cannot take place unless the medium is diluted, as the medium gels. On the other hand, good measurement reproducibility is observed for a dilution factor equal to 5.5.

With a test carried out on the same contaminated sample using a Petri dish put into an incubator at 30° C. according to the method well known to professionals in the field, formation of colonies of microorganisms begins to be visible to the naked eye at the end of 3 days.

The advantage of implementing the method according to the invention, allowing sample contamination to be revealed almost immediately, is thus demonstrated.

TABLE 2

| | Dilution | | | | | |
|---|---|---|---|---|---|---|
| | 480 | 240 | 10 | 5.5 | 2.8 | 0 |
| Relative quantity of light | 261,731 | 670,270 | 5,084,269 | 11,191,038 | 19,937,672 | Measurement |
| (URL) | | | | | | impossible |
| | 354,065 | 725,331 | 9,643,226 | 8,827,497 | 20,827,254 | — |
| | 435,682 | 473,199 | 6,866,485 | 9,756,096 | 11,550,343 | — |
| | 895,487 | 840,722 | 7,159,670 | 13,635,785 | 10909 798 | — |
| | 567,345 | 329,648 | 6,613,003 | 10,023,973 | 8,710,677 | — |
| | 256,786 | 608,158 | 13,019,536 | 14,139,563 | 1,307,613 | — |
| | 345 789 | 201,947 | 6,990,026 | 9,921,740 | 3,952,921 | — |
| Mean | 445,269 | 549,896 | 7,910,887 | 11,070,813 | 11,028,039 | — |
| Standard deviation | 225,505 | 226,826 | 2,622,869 | 2,048,900 | 7,366,671 | — |
| Relative standard deviation | 0.51 | 0.41 | 0.33 | 0.19 | 0.67 | — |

What we claim is:

1. A method for detecting a microorganism by bioluminescence comprising detecting said microorganism in an aqueous formulation containing a ASE or HASE-type polymer, wherein said method comprises implementing least one dilution of said aqueous formulation.

2. The method of claim 1, further comprising:
a) at least one dilution of the said aqueous formulation containing an ASE or HASE-type polymer,
b) contacting the formulation resulting from a) with at least one ATP-extracting compound and at least one pH-regulating compound,
c) contacting the medium resulting from b) with a luciferin/luciferase system,
d) measuring the quantity of light emitted by bioluminescence by the medium resulting from c).

3. The method of claim 2, wherein the dilution factor of the said aqueous formulation in a) ranges from 5 to 300.

4. The method of claim 2, wherein the dilution factor of the said aqueous formulation in a) ranges from 5 and 100

5. The method of claim 2, wherein the dilution factor of the said aqueous formulation in a) ranges from 5 and 20.

6. The method of claim 2, wherein said ATP-extracting compound is selected from the group consisting of an organic solvent and a surfactants.

7. The method of claim 2, wherein said ATP-extracting compound is trichloroacetic acid (TCA).

8. The method of claim 2, wherein said ATP-extracting compound is dimethyl sulfoxide (DMSO).

9. The method of claim 2, said pH-regulating agent is a buffer selected from the group consisting of an organic acid and an organic base.

10. The method of claim 2, said pH-regulating agent is a phosphate buffer comprising potassium or sodium dibasic phosphate.

11. The method of claim 2, wherein said dilution comprises mixing with a sterile aqueous solution.

12. The method of claim 11, wherein said sterile aqueous solution is a sterile isotonic aqueous solution.

13. The method of claim 11, wherein said sterile aqueous solution is a sterile isotonic aqueous peptone solution.

14. The method of claim 2, wherein said measuring comprises employing a luminometer having a probe comprising:
a) a tip for collection of a sample of the formulation to be analysed,
b) an ATP-extracting compound,
c) a pH-regulating compound,
d) a luciferin/luciferase system.

15. The method of claim 2, wherein said ASE-type polymer is a copolymer of (meth)acrylic acid with an ester of (meth)acrylic acid.

16. The method of claim 2, wherein said HASE-type polymer is a copolymer of (meth)acrylic acid, an ester of (meth)acrylic acid and a monomer with a hydrophobic group.

17. The method of claim 2, wherein said aqueous formulation is an emulsion, a dispersion or an aqueous composition containing a binder.

18. The method of claim 17, wherein said aqueous formulation is selected from the group consisting of a paper coating, a paint, a varnish, an ink, or a cosmetic or detergent composition.
